(19) United States
(12) Patent Application Publication
(10) Pub. No.: US 2018/0193304 A1
(43) Pub. Date: Jul. 12, 2018

(54) PHYTOTERPENOID FACILITATION OF THERAPEUTIC ONSET AND EFFICACY OF SUBLINGUAL CANNABINOID ADMINISTRATION

(71) Applicant: JC Pharma Inc., Bouder, CO (US)

(72) Inventors: Jason Allen Cranford, Boulder, CO (US); Donald Channing Cooper, Boulder, CO (US)

(73) Assignee: JC Pharma Inc., Boulder, CO (US)

(21) Appl. No.: 15/867,240

(22) Filed: Jan. 10, 2018

Related U.S. Application Data
(60) Provisional application No. 62/445,047, filed on Jan. 11, 2017.

(51) Int. Cl. A61K 31/352; A61K 31/05; A61K 31/015

(52) U.S. Cl. CPC A61K 31/352; A61K 31/015; A61K 31/05

(57) ABSTRACT

A cannabis plant extract based formulation to aid in stabilizing the therapeutic efficacy of cannabinoid containing treatments in patients affected with neurological diseases that comprises one or more of the following: Cannabidiol (CBD), tetrahydrocannabinol (THC), and terpenes; wherein the formulation comprises a high ratio of CBD to THC, with each of those cannabinoids in a relatively high concentration. The formulation also comprises Beta-caryophyllene that is used to further aid in neuroprotection by co-modulating CB1 and 2 receptors. Additionally, the formulation comprises the terpene humulene to assist in creating an “entourage effect” in conjunction with CBD, THC, and Beta-caryophyllene to stabilize and enhance treatment-related pharmacological actions. The formulation may also comprise Cannabichromene (CBC), Cannabigerol (CBG), and Cannabinol (CBN).

### CROSS REFERENCE TO RELATED APPLICATIONS

This application claims the benefit of U.S. Provisional Patent Application No. 62/2445,047, filed on Jan. 11, 2017, and titled “Cannabis Based Neuroprotection For Glutamate Excitotoxicity And Oxidative Stress” which is incorporated by reference herein in its entirety for all purposes.

### 1. FIELD OF THE INVENTION

The present invention is in the technical field of pharmaceutical compounds. More particularly, the present invention pertains to the field of pharmaceutical compounds and compositions that are useful as facilitators and co-modulators of phytocannabinoid-mediated pharmacological effects.

### 2. DESCRIPTION OF RELATED ART

Cannabis-based formulations have been used for centuries for the treatment of a variety of medical conditions. Although there are dozens of molecular variants of cannabinoids in a typical cannabis plant, the two major cannabinoids components 49 tetrahydrocannabinol (THC) and cannabidiol (CBD) have received the most attention for their medicinal effects. In 1997, the NIH reviewed scientific data concerning potential therapeutic uses for marijuana and hemp and found there may be beneficial medicinal effects and recommended that researchers develop alternative dosage forms other than smoking (NIDA, 1997). At least four clinical trials have examined cannabis compared with placebo for therapeutic uses (Abrams et al., 2007; Ellis et al. 2009; Ware et al., 2010; Wilsey et al., 2008). All four trials found a positive treatment effect with no serious adverse effects. Despite the wealth of data showing the medicinal effectiveness of cannabis and THC, the FDA has not approved the marijuana or hemp plant as medicine. The lack of US and European regulatory approval of cannabis containing products stemming from the stigma of marijuana use in some cultures and criminalization of marijuana possession may be responsible for the lack of standardized cannabinoid formulations and administration routes intended for the rational treatment of disease.

Over the last three decades scientific and medical research has gathered a substantial amount of evidence that phytocannabinoids and the associated phytochemicals present in Cannabis are useful in treating a variety of conditions including autism, seizures, cancer, multiple sclerosis among others. Although it is generally recognized that cannabis-associated phytochemicals are safe and therapeutically useful pharmacological agents, the exact pharmacodynamic signaling mechanisms and pharmacokinetic factors responsible for the wide variety of putative therapeutic effect is not fully understood. Following administration of cannabis containing formulations in humans, the central nervous system (CNS), peripheral nervous system (PNS) and immune systems variably respond to activation of cannabinoid CB1 and CB2 receptors that are variably distributed throughout the different systems.

CB1 and CB2 receptor signaling cascades work together synergistically or in opposition within different tissues depending on other co-factors present at the time of activation. Natural endogenous phytoterpenes present in the cannabis plant extracts are examples of such co-factors capable of co-modulating the pharmacological effects and physiological effects of CB1 and CB2 receptors. The major phytoterpenoids, such as alpha-pinene, linalool, beta caryophyllene, myrcene and limonene and their derivatives are capable of either enhancing or suppressing CB1 or CB2 pathways and possibly the absorption and metabolism/elimination of administered cannabinoids. This co-modulation of Cannabis extract by synergistically enhancing or modulating the effects of the cannabinoids is known as the “entourage effect”. Russo, E. B., (2011), Br J Pharmacol. August; 163(7). between cannabis phytoterpenes and cannabinoid signaling pathways provides a novel approach to fine tuning the CNS, PNS and immune systems and it can provide a method for improved treatment for pathologies that involve the endocannabinoid system.

Although there are dozens of active cannabinoids that have been described, the two major cannabinoids, tetrahydrocannabinol (THC) and cannabidiol (CBD) have received the most attention for their medicinal effects. The recent gradual legalization and decriminalization of marijuana over the past decade has led to a large increase in breeding and selection for high or low THC and CBD strains. As a consequence, the variability in the concentration of THC and CBD and the associated phytoterpenoids from plant extracts in the consumer marketplace has exponentially increased thus making reliable dosing for standard physiological treatment effects challenging. This has led to a variety of products on the market that claim to be beneficial for numerous pathologies. This variability in potency and relative ratios of cannabinoids and phytoterpenes in different products on the market has made it challenging for caregivers, physicians and those self-medicating to dose within optimal therapeutic ranges. To add to the confusion, cannabinoids may be smoked, vaporized, taken orally, sublingually, buccally or rectally with unacceptably wide variations in the rate of absorption, onset and duration of action. This contributes to the view shared by many Physicians that they are ill equipped to prescribe cannabinoids and/or marijuana for medical purposes.

In addition to the variability in cannabinoid potency and other additives within cannabis containing products throughout there are individual differences within treatment populations medicinally administering cannabinoids. Within the human endocannabinoid system developmental factors in children, genetic polymorphisms in metabolic pathways or receptor localization and signaling cascades can all differ between individuals and may be responsible for the wide range of effects of cannabis containing formulations amongst treatment populations.

The current state of the medicinal cannabis industry suffers from lack of standardization and regulation of formulations stemming from lack of coordinated regulatory oversight and sponsored research. In addition, the recent surge of proprietary cannabis containing products on the market with varying concentrations of unknown cannabinoids, additives and phytoterpene co-modulators has contributed to the variability in rational treatment solutions. Consequently, there is an immediate need for identification and standardization within the industry for formulations that have proven effectiveness across individuals and pathologies. As an example, the addition of phytoterpenoids, like beta-caryophyllene to phytocannabinoid extracts increases the agonist effect of CBD on CB receptors and act to co-modulate physiological functions in normal and pathological states. In some individuals the effects of cannabis containing medications lose their therapeutic effect over time and has been referred to a “honeymoon” period where the drug initially works well to manage symptoms but gradually becomes less effective. We have determined that the diminished therapeutic effect is due to non-optimal coordination between phytoterpenoid and cannabinoid signaling and/or pharmacokinetic co-modulation.

It is the purpose of this patent to address this problem and therefore, we offer a specific formulation approach that stabilizes the therapeutic effect of phytocannabinoids in a sublingual formulation containing specific ratios of THC:CBD and phytoterpenoids in treatment populations suffering from autism, seizures, cancer and other neurological and psychiatric disorders.

### DETAILED DESCRIPTION

The following description is not to be taken in a limiting sense, but is made merely for the purpose of describing the general principles of exemplary embodiments, many additional embodiments of this invention are possible. It is understood that no limitation of the scope of the invention is thereby intended. The scope of the disclosure should be determined with reference to the Claims. Reference throughout this specification to “one embodiment,” “an embodiment,” or similar language means that a particular feature, structure, or characteristic that is described in connection with the embodiment is included in at least one embodiment of the present disclosure. Thus, appearances of the phrases “in one embodiment,” “in an embodiment,” and similar language throughout this specification may, but do not necessarily, all refer to the same embodiment.

Further, the described features, structures, or characteristics of the present disclosure may be combined in any suitable manner in one or more embodiments. In the Detailed Description, numerous specific details are provided for a thorough understanding of embodiments of the disclosure. One skilled in the relevant art will recognize, however, that the embodiments of the present disclosure can be practiced without one or more of the specific details, or with other methods, components, materials, and so forth. In other instances, well-known structures, materials, or operations are not shown or described in detail to avoid obscuring aspects of the present disclosure. Any alterations and further modifications in the illustrated devices, and such further application of the principles of the invention as illustrated herein are contemplated as would normally occur to one skilled in the art to which the invention relates.

The phrases “at least one,” “one or more,” and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. The terms “a” or “an” entity refers to one or more of that entity. As such, the terms “a” (or “an”), “one or more” and “at least one” can be used interchangeably herein. It is also to be noted that the terms “comprising,” “including,” and “having” can be used interchangeably. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more and ending with a maximum value of 10 or less, e.g., 1 to 6.3, or 5.5 to 10, or 2.7 to 6.1.

Herein is disclosed a cannabis plant extract based formulation to aid in stabilizing the therapeutic efficacy of cannabinoid containing treatments in patients affected with neurological diseases that comprises one or more of the following: Cannabidiol (CBD), tetrahydrocannabinol (THC), and terpenes; wherein the formulation comprises a high ratio of CBD to THC, with each of those cannabinoids in a relatively high concentration. The formulation also comprises Beta-caryophyllene that is used to further aid in neuroprotection by co-modulating CB1 and 2 receptors. Additionally, the formulation comprises the terpene humulene to assist in creating an “entourage effect” in conjunction with CBD, THC, and Beta-caryophyllene to stabilize and enhance treatment-related pharmacological actions. The formulation may also comprise Cannabichromene (CBC), Cannabigerol (CBG), and Cannabinol (CBN).

In addition to cannabinoids, terpenes have been found to be helpful in providing CB2 activation. Caryophyllene is the only terpene known to interact with the endocannabinoid system (CB2). β-caryophyllene selectively binds to the CB2 receptor and is a functional CB2 agonist. Further, β-caryophyllene was identified as a functional non-psychoactive CB2 receptor ligand in foodstuff and as a macrocyclic anti-inflammatory cannabinoid in cannabis. (Proc Natl Acad Sci USA. 2008 Jul. 1; 105(26):9099-104. doi: 10.1073/pnas.0803601105. Epub 2008 Jun. 23. Beta-caryophyllene is a dietary cannabinoid.)

Many of the other cannabinoids, terpenoids, and flavonoids found in medical marijuana play a role in boosting the therapeutic effect of cannabis. The FDA and other agencies have generally recognized terpenes as “safe.”

Both humulene and caryophyllene displayed comparable anti-inflammatory responses to steroid alternatives. (Eur J Pharmacol. 2007 Aug. 27; 569(3):228-36. Epub 2007 May 22. Anti-inflammatory effects of compounds alpha-humulene and (−)-trans-caryophyllene isolated from the essential oil of Cordia verbenacea) Humulene was simultaneously effective in reducing inflammation and offering pain relief. (Planta Med. 2008 November; 74(14):1678-83. doi: 10.1055/s-0028-1088307. Epub 2008 Oct. 24. Pharmacokinetics and tissue distribution of the sesquiterpene alpha-humulene in mice.) The oral effects of humulene were analyzed and the results suggested that again, this terpene was highly effective at reducing inflammation, proving its usefulness as a topical or oral supplement. (Br J Pharmacol. 2009 October; 158(4): 1074-1087. Preventive and therapeutic anti-inflammatory properties of the sesquiterpene α-humulene in experimental airways allergic inflammation)

The formulation is designed to be administered to the user as a sublingual spray although it could be administered in a variety of ways, such as: in a pill, a drink, a tincture, etc.

The materials of the spray are primarily derived from cannabis plant material. The cannabis is preferably grown using organic methods and extracted from the plant using carbon dioxide (CO2) extraction methods. It is well understood that there are many ways to grow cannabis and make extracts therefrom, and this patent is intended to cover all such methods. The cannabis oil extraction may then be purified using a short path distiller and, optionally, a winterization process that removes plant wax and contaminants. The cannabis extract may then be purged of all solvents and decarboxylated using a vacuum purge oven. The extract may then be decarboxylated while covered to prevent terpene loss. The plant extract can then be tested for potency, microbial and pesticide contamination, terpenes, residual solvents, and potency using High Performance Liquid Chromatography (HPLC) and/or gas chromatography (GC). The plant extract may then be infused into an organic botanical oil carrier liquid; possibly using a hot plate, a top loading milligram scale, and a hand held ultrasonic homogenizer until the liquid is roughly even in color and consistency. The product may then tested again for final potency of cannabinoid and terpenes using High Performance Liquid Chromatography (HPLC) and/or gas chromatography (GC). The final product may then be put into an oral spray device with a bottle pipette that measures the volume in each spray bottle. The oral spray may also use a metered atomizer to dispense a standardized amount of 0.16 ml per spray of cannabinoid infused liquid to the user. One spray is intended to supply 5 mg of THC and 1.47 mg of CBD. The intended ratio is roughly 1:4 CBD:THC.

The preferred formulation is as follows:

Cannabidiol 14.77%

Tetrahydrocannabinol 50.04%

Cannabichromene 1.07%

Cannabigerol 3.61%

Cannabinol 0.97%

Beta-caryophyllene 20.43%

Humulene 1.14%

Carrier; wherein the carrier liquid is an unflavored, unscented non-GMO certified, gluten free, medium chain triglycerides coconut oil.

The formulation need not have the proportions as outlined in the preferred embodiment to be effective. The following ranges of ingredients are also effective at creating the desired effects:

Cannabidiol 8% to 25%

Tetrahydrocannabinol 30% to 70%

Wherein the ratio of Tetrahydrocannabinol to Cannabidiol is between 3:1 and 5:1

Cannabichromene 0% to 4%

Cannabigerol 0% to 7%

Cannabinol 0% to 4%

Beta-caryophyllene 10% to 30%

Humulene 0% to 3%

Carrier liquid (any) 0%-20%

Information as herein shown and described in detail is fully capable of attaining the above-described object of the present disclosure, the presently preferred embodiment of the present disclosure; and is, thus, representative of the subject matter; which is broadly contemplated by the present disclosure. The scope of the present disclosure fully encompasses other embodiments which may become obvious to those skilled in the art, and is to be limited, accordingly, by nothing other than the appended claims, wherein any reference to an element being made in the singular is not intended to mean “one and only one” unless explicitly so stated, but rather “one or more.” All structural and functional equivalents to the elements of the above described preferred embodiment and additional embodiments as regarded by those of ordinary skill in the art are hereby expressly incorporated by reference and are intended to be encompassed by the present claims.

Moreover, no requirement exists for a system or method to address each and every problem sought to be resolved by the present disclosure, for such to be encompassed by the present claims. Furthermore, no element, component, or method step in the present disclosure is intended to be dedicated to the public regardless of whether the element, component, or method step is explicitly recited in the claims. However, that various changes and modifications in form, material, work-piece, and fabrication material detail may be made, without departing from the spirit and scope of the present disclosure, as set forth in the appended claims, as may be apparent to those of ordinary skill in the art, are also encompassed by the present disclosure.

What is claimed is:

1. A formulation, comprising:
at least 30% tetrahydrocannabinol;
at least 8% cannabidiol; and
at least 10% beta-caryophyllene.

2. The formulation of claim 1 further comprising at least 0.5% humulene.

3. The formulation of claim 1 wherein the ratio of tetrahydrocannabinol to cannabidiol is between 3:1 and 5:1.

4. The formulation of claim 1 further comprising cannabichromene.

5. The formulation of claim 1 further comprising cannabigerol.

6. The formulation of claim 1 further comprising cannabinol.

7. The formulation of claim 1 further comprising a carrier.
